SEQUENCE LISTING (16 sequences)

SEQ ID NO: 1 - DNA, 24 bp, Artificial Sequence
  Primer
atgcctgcaccaaaactcacggag

SEQ ID NO: 2 - DNA, 24 bp, Artificial Sequence
  Primer
ctacgcatctccttctttcccttc

SEQ ID NO: 3 - DNA, 24 bp, Artificial Sequence
  Primer
atgtctgctcccgctgccgatcat

SEQ ID NO: 4 - DNA, 24 bp, Artificial Sequence
  Primer
tcattctttcttttcgtgttctct

SEQ ID NO: 5 - DNA, 2232 bp, Saccharomyces cerevisiae
  CDS (1)..(2232)
atgtctgctcccgctgccgatcataacgctgccaaacctattcctcatgtacctcaagcgtcccgacggtacaaaaattcatacaatggattcgtatacaatatacatacatggctgtatgatgtgtctgtatttctgtttaatattttgttcactattttcttcagagaaattaaggtacgtggtgcatataacgttcccgaagttggggtgccaaccatccttgtgtgtgcccctcatgcaaatcagttcatcgacccggctttggtaatgtcgcaaacccgtttgctgaagacatcagcgggaaagtcccgatccagaatgccttgttttgttactgctgagtcgagttttaagaaaagatttatctctttctttggtcacgcaatgggcggtattcccgtgcctagaattcaggacaacttgaagccagtggatgagaatcttgagatttacgctccggacttgaagaaccacccggaaatcatcaagggccgctccaagaacccacagactacaccagtgaactttacgaaaaggttttctgccaagtccttgcttggattgcccgactacttaagtaatgctcaaatcaaggaaatcccggatgatgaaacgataatcttgtcctctccattcagaacatcgaaatcaaaagtggtggagctcttgactaatggtactaattttaaatatgcagagaaaatcgacaatacggaaactttccagagtgtttttgatcacttgcatacgaagggctgtgtaggtattttccccgagggtggttctcatgaccgtccttcgttactacccatcaaggcaggtgttgccattatggctctgggcgcagtagccgctgatcctaccatgaaagttgctgttgtaccctgtggtttgcattatttccacagaaataaattcagatctagagctgttttagaatacggcgaacctatagtggtggatgggaaatatggcgaaatgtataaggactccccacgtgagaccgtttccaaactactaaaaaagatcaccaattctttgttttctgttaccgaaaatgctccagattacgatactttgatggtcattcaggctgccagaagactatatcaaccggtaaaagtcaggctacctttgcctgccattgtagaaatcaacagaaggttacttttcggttattccaagtttaaagatgatccaagaattattcacttaaaaaaactggtatatgactacaacaggaaattagattcagtgggtttaaaagaccatcaggtgatgcaattaaaaactaccaaattagaagcattgaggtgctttgtaactttgatcgttcgattgattaaattttctgtctttgctatactatcgttaccgggttctattctcttcactccaattttcattatttgtcgcgtatactcagaaaagaaggccaaagagggtttaaagaaatcattggttaaaattaagggtaccgatttgttggccacatggaaacttatcgtggcgttaatattggcaccaattttatacgttacttactcgatcttgttgattattttggcaagaaaacaacactattgtcgcatctgggttccttccaataacgcattcatacaatttgtctatttttatgcgttattggttttcaccacgtattcctctttaaagaccggtgaaatcggtgttgaccttttcaaatctttaagaccactttttgtttctattgtttaccccggtaagaagatcgaagaaatccaaacaacaagaaagaatttaagtctagagttgactgctgtttgtaacgatttaggacctttggttttccctgattacgataaattagcgactgagatattctctaagagagacggttatgatgtctcttctgatgcagagtcttctataagtcgtatgagtgtacaatctagaagccgctcttcttctatacattctattggctcgctagcttctaacgccctatcaagagtgaattcaagaggctcgttgaccgatattccaattttttctgatgcaaagcaaggtcaatggaaaagtgaaggtgaaactagtgaggatgaggatgaatttgatgagaaaaatcctgccatagtacaaaccgcacgaagttctgatctaaataaggaaaacagtcgcaacacaaatatatcttcgaagattgcttcgctggtaagacagaaaagagaacacgaaaagaaagaatga

SEQ ID NO: 6 - protein, 743 aa, Saccharomyces cerevisiae
MSAPAADHNAAKPIPHVPQASRRYKNSYNGFVYNIHTWLYDVSVFLFNILFTIFFREIKVRGAYNVPEVGVPTILVCAPHANQFIDPALVMSQTRLLKTSAGKSRSRMPCFVTAESSFKKRFISFFGHAMGGIPVPRIQDNLKPVDENLEIYAPDLKNHPEIIKGRSKNPQTTPVNFTKRFSAKSLLGLPDYLSNAQIKEIPDDETIILSSPFRTSKSKVVELLTNGTNFKYAEKIDNTETFQSVFDHLHTKGCVGIFPEGGSHDRPSLLPIKAGVAIMALGAVAADPTMKVAVVPCGLHYFHRNKFRSRAVLEYGEPIVVDGKYGEMYKDSPRETVSKLLKKITNSLFSVTENAPDYDTLMVIQAARRLYQPVKVRLPLPAIVEINRRLLFGYSKFKDDPRIIHLKKLVYDYNRKLDSVGLKDHQVMQLKTTKLEALRCFVTLIVRLIKFSVFAILSLPGSILFTPIFIICRVYSEKKAKEGLKKSLVKIKGTDLLATWKLIVALILAPILYVTYSILLIILARKQHYCRIWVPSNNAFIQFVYFYALLVFTTYSSLKTGEIGVDLFKSLRPLFVSIVYPGKKIEEIQTTRKNLSLELTAVCNDLGPLVFPDYDKLATEIFSKRDGYDVSSDAESSISRMSVQSRSRSSSIHSIGSLASNALSRVNSRGSLTDIPIFSDAKQGQWKSEGETSEDEDEFDEKNPAIVQTARSSDLNKENSRNTNISSKIASLVRQKREHEKKE

SEQ ID NO: 7 - DNA, 2253 bp, Saccharomyces cerevisiae
  CDS (1)..(2253)
atgcctgcaccaaaactcacggagaaatctgcctcttccaagagcacacagaaaactacgaattacagttccatcgaggccaaaagcatctaccaagagcctagcgctaccaagaagatactttactccatcgccacatggctgttgtacaacatcttccactgcttctttagagaaatcagaggccggggcagtttcaaggtaccgcaacagggaccggtgatctttgttgcggctccgcatgctaaccagttcgtcgaccctgtaatccttatgggcgaggtgaagaaatctgtcaacagacgtgtgtccttcttgattgcggagagctcattaaagcaaccccccatagggtttttggctagtttcttcatggccataggcgtggtaaggccgcaggataatttgaaaccggcagaaggtactatccgcgtagatccaacagactacaagagagttatcggccacgacacgcatttcttgactgattgtatgccaaagggtctcatcgggttacccaaatcaatgggatttggagaaatccagtccatagaaagtgacacgagtttgaccctaagaaaagagttcaaaatggccaaaccagagattaaaactgctttactcaccggcactacttataaatatgccgctaaagtcgaccaatcttgcgtttaccatagagtttttgagcatttggcccataacaactgcattgggatctttcctgaaggtgggtcccacgacagaacaaacttgttgcccctgaaagcaggtgtggcgattatggctcttggttgcatggataggcatcctgacgtcaatgttaagattgttccctgcggtatgaattatttccatccacataagttcaggtcgagagcggttgttgaattcggtgaccccattgaaataccgaaggaactagtcgccaagtaccacaactcggaaacgaacagagatgcagtgaaagaattattagataccatatcgaagggtttacaatccgttaccgttacatgttctgattatgaaactttgatggtggttcaaacgataagaagactatatatgacacaatttagcaccaagttaccgttgcccttgattgtggaaatgaacagaagaatggtcaaaggttacgaattctatagaaacgatcctaaaatagcggacttgaccaaagatataatggcatataatgccgccttgagacactataatcttcctgatcaccttgtggaggaggcaaaggtaaatttcgcaaaaaacctcggacttgttttttttagatccatcgggctctgcatcctcttttcgttagccatgccaggtatcattatgttctcacctgtcttcatattagccaagagaatttctcaagaaaaggcccgtaccgctttgtccaagtctacagttaaaataaaggctaacgatgtcattgccacgtggaaaatcttgattgggatgggatttgcgcccttgctttacatcttttggtccgttttaatcacttattacctcagacataaaccatggaataaaatatatgttttttccgggtcttacatctcgtgtgttatagtcacgtattccgccttaatcgtgggtgatattggtatggatggtttcaaatctttgagaccactggttttatctcttacatctccaaagggcttgcaaaagctacaaaaaaatcgtagaaatctggcagaaagaataatcgaagttgtaaataactttggaagcgaattattccccgatttcgatagtgccgccctacgtgaagaattcgacgtcatcgatgaagaggaagaagatcgaaaaacctcagaattgaatcgcaggaaaatgctaagaaaacagaaaataaaaagacaagaaaaagattcgtcatcacctatcatcagccaacgtgacaaccacgatgcctatgaacaccataaccaagattccgatggcgtctcattggtcaatagtgacaattccctctctaacattccattattctcttctacttttcatcgtaagtcagagtcttccttagcttcgacatccgttgcaccttcttcttcctccgaatttgaggtagaaaacgaaatcttggaggaaaaaaatggattagcaagtaaaatcgcacaggccgtcttaaacaagagaattggtgaaaatactgccagggaagaggaagaggaagaagaagaggaagaagaagaagaggaagaagaagaagaagggaaagaaggagatgcgtag

SEQ ID NO: 8 - protein, 750 aa, Saccharomyces cerevisiae
MPAPKLTEKSASSKSTQKTTNYSSIEAKSIYQEPSATKKILYSIATWLLYNIFHCFFREIRGRGSFKVPQQGPVIFVAAPHANQFVDPVILMGEVKKSVNRRVSFLIAESSLKQPPIGFLASFFMAIGVVRPQDNLKPAEGTIRVDPTDYKRVIGHDTHFLTDCMPKGLIGLPKSMGFGEIQSIESDTSLTLRKEFKMAKPEIKTALLTGTTYKYAAKVDQSCVYHRVFEHLAHNNCIGIFPEGGSHDRTNLLPLKAGVAIMALGCMDRHPDVNVKIVPCGMNYFHPHKFRSRAVVEFGDPIEIPKELVAKYHNSETNRDAVKELLDTISKGLQSVTVTCSDYETLMVVQTIRRLYMTQFSTKLPLPLIVEMNRRMVKGYEFYRNDPKIADLTKDIMAYNAALRHYNLPDHLVEEAKVNFAKNLGLVFFRSIGLCILFSLAMPGIIMFSPVFILAKRISQEKARTALSKSTVKIKANDVIATWKILIGMGFAPLLYIFWSVLITYYLRHKPWNKIYVFSGSYISCVIVTYSALIVGDIGMDGFKSLRPLVLSLTSPKGLQKLQKNRRNLAERIIEVVNNFGSELFPDFDSAALREEFDVIDEEEEDRKTSELNRRKMLRKQKIKRQEKDSSSPIISQRDNHDAYEHHNQDSDGVSLVNSDNSLSNIPLFSSTFHRKSESSLASTSVAPSSSSEFEVENEILEEKNGLASKIAQAVLNKRIGENTAREEEEEEEEEEEEEEEEEEGKEGDA

SEQ ID NO: 9 - DNA, 33 bp, Artificial Sequence
  Primer
ggatccaacatgtctgctcccgctgccgatcat

SEQ ID NO: 10 - DNA, 30 bp, Artificial Sequence
  Primer
ctcgagtcattctttcttttcgtgttctct

SEQ ID NO: 11 - DNA, 33 bp, Artificial Sequence
  Primer
ggatccaacatgcctgcaccaaaactcacggag

SEQ ID NO: 12 - DNA, 30 bp, Artificial Sequence
  Primer
ctcgagctacgcatctccttctttcccttc

SEQ ID NO: 13 - protein, 55 aa, Escherichia coli
ELFSRGYSVEYFVEGGRSRTGRLLDPKTGTLSMTIQAMLRGGTRPITLIPIYIGY

SEQ ID NO: 14 - protein, 55 aa, Rattus norvegicus
ELLRQQQFLEIFLEGTRSRSGKTSCARAGLLSVVVDTLSSNTIPDILVIPVGISY

SEQ ID NO: 15 - protein, 55 aa, Saccharomyces cerevisiae
EHLAHNNCIGIFPEGGSHDRTNLLPLKAGVAIMALGCMDRHPDVNVKIVPCGMNY

SEQ ID NO: 16 - protein, 55 aa, Saccharomyces cerevisiae
DHLHTKGCVGIFPEGGSHDRPSLLPIKAGVAIMALGAVAADPTMKVAVVPCGLHY

(19) United States
(12) Patent Application Publication
(10) Pub. No.: US 2004/0043449 A1
(43) Pub. Date: Mar. 4, 2004

(54) Glycerol-3-phosphate/dihydroxyacetone phosphate dual substrate acyltransferases

(76) Inventors: Jitao Zou, Saskatoon (CA); Zhifu Zheng, Saskatoon (CA)

Correspondence Address: KIRBY EADES GALE BAKER, BOX 3432, STATION D, OTTAWA, ON, K1P 6N9, CA

(21) Appl. No.: 10/332,995

(22) PCT Filed: Jul. 25, 2001
(86) PCT No.: PCT/CA01/01073

(51) Int. Cl. C07H 21/04; C12N 9/10; C12P 21/02; C12N 5/06

(52) U.S. Cl. 435/69.1; 435/193; 435/320.1; 435/325; 536/23.2

(57) ABSTRACT

The present invention relates to DNA sequences encoding novel Glycerol-3-Phosphate and Dihydroxy Acetone Phosphate acyltransferase enzymes, and methods for utilizing said polynucleotides for alteration of lipid content in higher cells, in particular plant cells, as well as other purposes.

### TECHNICAL FIELD

The present invention relates to Glycerol-3-Phosphate and Dihydroxy Acetone Phosphate acyltransferase enzymes, methods to identify and isolate polynucleotides encoding said enzymes and methods for utilizing said polynucleotides for alteration of lipid content in higher cells, and for other purposes.

### BACKGROUND ART

It widely known that phospholipids play a major role as structural elements in membranes and as cell signaling components and numerous studies have illustrated the role of lipids in a variety of cellular processes (e.g., Daum, G., et al., (1998) Yeast 14, 1471-1510; Carman, G. M., and Henry, S. A. (1999) Prog. Lipid Res. 38, 361-399; Moolenaar, W. H. (1995) J. Biol. Chem. 270,12949-12952; English, D., et al., (1996) Chem Phys Lipids 80, 117-132). However, there are still significant knowledge gaps with regard to various aspects of regulation of the phospholipid biosynthetic pathway (Duam, ibid; Carmen and Henry, ibid). Understanding the biosynthesis of lipid formation, the controlling steps and the interactions between various steps in the pathway represent crucial information needed for the directed modification of lipid content in cells. Although the lipid biosynthetic pathway contains numerous branch points, lipid biosynthesis typically occurs with the initial step of acylation of G-3-P (Glycerol-3-Phosphate) at the sn-1 position by a G-3-P acyltransferase to form lysophosphatidic acid (LPA). An alternative path for the formation of lysophosphatidic acid is the esterification of a fatty acyl group with Dihydroxyacetone Phosphate (DHAP). In this pathway, DHAP and fatty acyl-CoA are acted upon by the enzyme Dihydroxyacetone-phosphate acyltransferase to form fatty acyl dihydroxyphosphate which in the presence of NADPH can be converted to lysophosphatidic acid.

LPA acyltransferase then catalyzes the acylation of LPA at the sn-2 position to generate phosphatidic acid (PA), which serves as a general precursor for all glycerophospholipids and, in eukaryotes, triacylglycerol (Dircks, L. and Sul, H. S. (1999). Prog. Lipid Res. 38, 461-479; Christiansen, K (1978) Biochim. Biophys. Acta. 530(1), 78-90). In Escherichia coli, an integral membrane protein (p1sB) is responsible for the G-3-P acyltransferase activity, and its corresponding gene has been identified (Wilkison, W. O., and Bell, R. M. (1997) Biochim. Biophys. Acta. 1348, 3-9). In eukaryotic cells, multiple isoforms of G-3-P acyltransferase are present and localized in different intracellular compartments (Dircks, L. K., and Sul, H. S. (1997) Biochim. Biophys. Acta. 1348, 17-26; Murata, N., and Tasaka, Y. (1997) Biochim. Biophys. Acta. 1348, 10-16). The genes corresponding to the mammalian mitochondrial and plant plastidial localized G-3-P acyltransferase have been isolated and characterized in detail (Dircks, ibid., Murata, ibid.). In contrast, the eukaryotic microsomal counterpart has so far remained elusive, mainly due to the difficulties encountered in the purification of these membrane proteins and reconstitution of functional enzymes.

Accordingly, little is known about the structure or specific activity of G-3-P acyltransferases that are not mitochondrial or plastid localized to mitochondria or plastids. Similarly little is known about the structure or specific activity of the Dihydroxyacetone-phosphate (DHAP) acyltransferase enzyme in higher organisms.

Baker's yeast, Saccharomyces cerevisiae, is a convenient model organism for eukaryotic lipid studies since its glycerolipid biosynthetic pathway is highly similar to a wide spectrum of species including higher plants and mammals. More than a decade ago, Tillman and Bell reported mutants deficient in the activities of G-3-P acyltransferase in Saccharomyces cerevisiae (Tillman, T. S., and Bell, R. M. (1986) J. Biol. Chem. 261(20), 9144-9). Subsequent biochemical characterizations of one such mutant, generally known as TTA1, have yielded many new insights into lipid metabolism. Based on anlysis of this mutant, it is now widely accepted that the initial step of glycerolipid biosynthesis in yeast is mediated by a G-3-P/DHAP dual substrate acyltransferase (Tillman, ibid., Athenstaedt, K., et al., (1999) J. Bacteriol. 181(5), 1458-63), and that multiple isoforms of G-3-P acyltransferase are present in yeast (Athenstaedt, ibid., Athenstaedt, K., and Daum, G. (1997) J. Bacteriol. 179(24), 7611-6). However, the protein and the gene corresponding to the mutation have not been identified due to the lack of an apparent selectable growth phenotype in TTA1. Little is known about these enzymes in other organisms.

Thus, the gene structure and function of the cytoplasmic forms of G-3-P actyltransferase/DHAP acyltransferase has not been clearly identified in higher organisms and little is known about the structure and regulation of this enzyme.

Attempts to modify oil composition and content in higher organisms, in particular plants require an understanding of the mechanisms by which oil content is regulated. The enzymes G-3-P acyltransferase and DHAP acyltransferase represent initial steps in lipid biosynthesis and triglyceride formation, thus manipulation of the enzyme levels or the activity of these enzymes can be anticipated to cause major alteration of lipid content in cells. Although there are many known lipid enzyme activities, the cytoplasmic localized G-3-P acyltransferase and DHAP acyltransferase enzymes are poorly understood, and the nature of the genes encoding said enzymes and the structure of the enzymes themselves not particularly well-characterized. These two enzymatic activities represent one of the first steps in the assembly of triglycerides in higher organisms. Both activities lead to the formation of lysophosphatidic acid, which is the primary precursor for the formation of phosphatidic acid and diacylglycerol. Diacylglycerol feeds into the Kennedy pathway leading to the formation of triglycerides. Understanding the structure and activity of these enzymes can be used for the design of strategies for alteration of lipid content.

It is expected that over expression of these enzymes can lead to hyper-accumulation of lysophosphatidic acid which in turn can lead to increased levels of triglycerides or alterations in lipid composition. Thus, control of the initial step of the formation of lysophosphatidic acid can be expected to lead to significant changes in lipid composition and concentration.

Furthermore, when the expression of these enzymes is reduced, it is anticipated that reduced levels of triglycerides or lipids can be expected.

Accordingly, the isolation of the genes that encode these enzymes can provide the nucleic acids to construct genetic constructs capable of causing an alteration in the activity of these two enzymes in a variety of organisms. It is for this purpose that these genes are considered key components in strategies aimed at manipulation of lipid content and composition, particularly of plants (and especially oilseed plants, such as rape and canola).

### DISCLOSURE OF THE INVENTION

The present invention provides nucleic acid sequences derived from Saccharomyces cerevisiae encoding dual substrate acyltransferases capable of utilizing both Glycerol-3-Phosphate and Dihydroxy Acetone Phosphate as acyl acceptors. The sequences are useful for modification of lipid composition in higher organisms and can be used for isolation of similar enzymatic activities from other organisms. The invention further relates to the enzymes encoded by said nucleic acids and the use thereof.

Thus, according to one aspect of the invention, there is provided an isolated polynucleotide comprising: a nucleotide sequence of SEQ ID NO:5 or SEQ ID NO:7, or the complementary strand of said sequence; or a polynucleotide sequence that hybridizes under stringent conditions to the protein coding regions of SEQ ID NO:5 or SEQ ID NO:7, or their complementary strands or fragments thereof; or a polynucleotide sequence which, but for the degeneracy of the genetic code, would hybridize under stringent conditions to the polynucleotide sequence of SEQ ID NO:5 or SEQ ID NO:7.

According to another aspect of the invention, there is provided a polypeptide having an amino acid sequence according to SEQ ID NO:6 or SEQ ID NO:8, or having a sequence with at least 80% identity thereto.

In another aspect of the invention, nucleic acid sequences are provided that encode a G-3-P acyltransferase enzyme named Gat1p that is a sn-1 fatty acyltransferase that represents an enzyme found primarily in the cytoplasm associated with lipid particles and has nearly equal preference for DHAP and G-3-P as substrates for acylation.

In yet another aspect of the present invention, nucleotide sequences are provided that encode a G-3-P acyltransferase enzyme named Gat2p that is a sn-1 fatty acyltransferase that represents a membrane associated enzyme found primarily in association with cytoplasm membranes that has a preference for G-3-P over DHAP as a substrate for acylation.

In still another aspect of the present invention methods are described that enable the heterologous expression of said enzymes in a host cell.

According to yet another aspect of the invention, there is provided a method of modifying the lipid composition of a cell comprising: introducing into a cell capable of being transformed a genetic construct comprising a first DNA expression cassette that comprises, in addition to DNA sequences required for transformation and selection in said cells, a polynucleotide according to any one of claims 1 to 8, operably linked to a transcriptional regulatory region; and recovering a cell which contains said genetic construct.

According to yet another aspect of the invention, there is provided a method of identifying Glycerol-3-Phosphate acyltransferase (G-3-P) or Dihyroxy Acetone Phosphate acyltransferase (DHAP) genes comprising: (a) producing a cell comprising a conditional choline auxotrophic lipid mutant, wherein growth of said mutant is inhibited by high levels of inositol, said mutant being capable of suppression by supplementation of choline to an inositol-containing medium; (b) producing, as a second mutant, a choline transporter mutant; (c) combining the first and second mutants to form a double mutant; and (d) screening said double mutant with cloned DNA, modified for expression in said cell, to identify a G-3-P/DHAP acyltransferase encoded genes capable or restoring normal growth to said mutants.

The preferred DNA sequences to which the present invention relates are set out below and also in the accompanying Sequence Listing.

SEQ ID NO:5:
Sequence of YBL011w DNA
5′-ATGTCTGCTCCCGCTGCCGATCATAACGCTGCCAAAC
CTATTCCTCATGTACCTCAAGCGTCCCGACGGTACAAAAATTCATACAATGGATTCGTATACAATATACA
TACATGGCTGTATGATGTGTCTGTATTTCTGTTTAATATTTTGTTCACTATTTTCTTCAGAGAAATTAAG
GTACGTGGTGCATATAACGTTCCCGAAGTTGGGGTGCCAACCATCCTTGTGTGTGCCCCTCATGCAAATC
AGTTCATCGACCCGGCTTTGGTAATGTCGCAAACCCGTTTGCTGAAGACATCAGCGGGAAAGTCCCGATC
CAGAATGCCTTGTTTTGTTACTGCTGAGTCGAGTTTTAAGAAAAGATTTATCTCTTTCTTTGGTCACGCA
ATGGGCGGTATTCCCGTGCCTAGAATTCAGGACAACTTGAAGCCAGTGGATGAGAATCTTGAGATTTACG
CTCCGGACTTGAAGAACCACCCGGAAATCATCAAGGGCCGCTCCAAGAACCCACAGACTACACCAGTGAA
CTTTACGAAAAGGTTTTCTGCCAAGTCCTTGCTTGGATTGCCCGACTACTTAAGTAATGCTCAAATCAAG
GAAATCCCGGATGATGAAACGATAATCTTGTCCTCTCCATTCAGAACATCGAAATCAAAAGTGGTGGAGC
TCTTGACTAATGGTACTAATTTTAAATATGCAGAGAAAATCGACAATACGGAAACTTTCCAGAGTGTTTT
TGATCACTTGCATACGAAGGGCTGTGTAGGTATTTTCCCCGAGGGTGGTTCTCATGACCGTCCTTCGTTA
CTACCCATCAAGGCAGGTGTTGCCATTATGGCTCTGGGCGCAGTAGCCGCTGATCCTACCATGAAAGTTG
CTGTTGTACCCTGTGGTTTGCAATTATTTCCACAGAATAAATTCAGATCTAGAGCTGTTTTAGAATACGG
CGAACCTATAGTGGTGGATGGGAAATATGGCGAAATGTATAAGGACTCCCCACGTGAGACCGTTTCCAAA
CTACTAAAAAAGATCACCAATTCTTTGTTTTCTGTTACCGAAAATGCTCCAGATTACGATACTTTGATGG
TCATTCAGGCTGCCAGAAGACTATATCAACCGGTAAAAGTCAGGCTACCTTTGCCTGCCATTGTAGAAAT
CAACAGAAGGTTACTTTTCGGTTATTCCAAGTTTAAAGATGATCCAAGAATTATTCACTTAAAAAAACTG
GTATATGACTACAACAGGAAATTAGATTCAGTGGGTTTAAAAGACCATCAGGTGATGCAATTAAAAACTA
CCAAATTAGAAGCATTGAGGTGCTTTGTAACTTTGATCGTTCGATTGATTAAATTTTCTGTCTTTGCTAT
ACTATCGTTACCGGGTTCTATTCTCTTCACTCCAATTTTCATTATTTGTCGCGTATACTCAGAAAAGAAG
GCCAAAGAGGGTTTAAAGAAATCATTGGTTAAAATTAAGGGTACCGATTTGTTGGCCACATGGAAACTTA
TCGTGGCGTTAATATTGGCACCAATTTTATACGTTACTTACTCGATCTTGTTGATTATTTTGGCAAGAAA
ACAACACTATTGTCGCATCTGGGTTCCTTCCAATAACGCATTCATACAATTTGTCTATTTTTATGCGTTA
TTGGTTTTCACCACGTATTCCTCTTTAAAGACCGGTGAAATCGGTGTTGACCTTTTCAAATCTTTAAGAC
CACTTTTTGTTTCTATTGTTTACCCCGGTAAGAAGATCGAAGAAATCCAAACAACAAGAAAGAATTTAAG
TCTAGAGTTGACTGCTGTTTGTAACGATTTAGGACCTTTGGTTTTCCCTGATTACGATAAATTAGCGACT
GAGATATTCTCTAAGAGAGACGGTTATGATGTCTCTTCTGATGCAGAGTCTTCTATAAGTCGTATGAGTG
TACAATCTAGAAGCCGCTCTTCTTCTATACATTCTATTGGCTCGCTAGCTTCTAACGCCCTATCAAGAGT
GAATTCAAGAGCTCGTTGACCGATATTCCAATTTTTTTCTGATGCAAAGCAAGGTCAATGGAAAAGTGAA
GGTGAAACTAGTGAGGATGAGGATGAATTTGATGAGAAAAATCCTGCCATAGTACAAACCGCACGAAGTT
CTGATCTAAATAAGGAAAACAGTCGCAACACAAATATATCTTCGAAGATTGCTTCGCTGGTAAGACAGAA
AAGAGAACACGAAAAGAAAGAATGA-3′.
SEQ ID NO:7:
Sequence of YKR067w DNA
5′-ATGCCTGCACCAAAACTCACGGAGAAATCTGCCTCTTCCAAGAGCACACAGAAAACTACGAATTACA
GTTCCATCGAGGCCAAAAGCATCTACCAAGAGCCTAGCGCTACCAAGAAGATACTTTACTCCATCGCCAC
ATGGCTGTTGTACAACATCTTCCACTGCTTCTTTAGAGAAATCAGAGGCCGGGGCAGTTTCAAGGTACCG
CAACAGGGACCGGTGATCTTTGTTGCGGCTCCGCATGCTAACCAGTTCGTCGACCCTGTAATCCTTATGG
GCGAGGTGAAGAAATCTGTCAACAGACGTGTGTCCTTCTTGATTGCGGAGAGCTCATTAAAGCAACCCCC
CATAGGGTTTTTGGCTAGTTTCTTCATGGCCATAGGCGTGGTAAGGCCGCAGGATAATTTGAAACCGGCA
GAAGGTACTATCCGCGTAGATCCAACAGACTACAAGAGAGTTATCGGCCACGACACGCATTTCTTGACTG
ATTGTATGCCAAAGGGTCTCATCGGGTTACCCAAATCAATGGGATTTGGAGAAATCCAGTCCATAGAAAG
TGACACGAGTTTGACCCTAAGAAAAGAGTTCAAAATGGCCAAACCAGAGATTAAAACTGCTTTACTCACC
GGCACTACTTATAAATATGCCGCTAAAGTCGACCAATCTTGCGTTTACCATAGAGTTTTTGAGCATTTGG
CCCATAACAACTGCATTGGGATCTTTCCTGAAGGTGGGTCCCACGACAGAACAAACTTGTTGCCCCTGAA
AGCAGGTGTGGCGATTATGGCTCTTGGTTGCATGGATAGGCATCCTGACGTCAATGTTAAGATTGTTCCC
TGCGGTATGAATTATTTCCATCCACATAAGTTCAGGTCGAGAGCGGTTGTTGAATTCGGTGACCCCATTG
AAATACCGAAGGAACTAGTCGCCAAGTACCACAACTCGGAAACGAACAGAGATGCAGTGAAAGAATTATT
AGATACCATATCGAAGGGTTTACAATCCGTTACCGTTACATGTTCTGATTATGAAACTTTGATGGTGGTT
CAAACGATAAGAAGACCTAATATGACACAATTTAGCACCAAGTTACCGTTGCCCTTGATTGTGGAAATGA
ACAGAAGAATGGTCAAAGGTTACGAATTCTATAGAAACGATCCTAAAATAGCGGACTTGACCAAAGATAT
AATGGCATATAATGCCGCCTTGAGACACTATAATCTTCCTGATCACCTTGTGGAGGAGGCAAAGGTAAAT
TTCGCAAAAAACCTCGGACTTGTTTTTTTTAGATCCATCGGGCTCTGCATCCTCTTTTCGTTAGCCATGC
CAGGTATCATTATGTTCTCACCTGTCTTCATATTAGCCAAGAGAATTTCTCAAGAAAAGGCCCGTACCGC
TTTGTCCAAGTCTACAGTTAAAATAAAGGCTAACGATGTCATTGCCACGTGGAAAATCTTGATTGGGATG
GGATTTGCGCCCTTGCTTTACATCTTTTGGTCCGTTTTAATCACTTATTACCTCAGACATAAACCATGGA
ATAAAATATATGTTTTTTCCGGGTCTTACATCTCGTGTGTTATAGTCACGTATTCCGCCTTAATCGTGGG
TGATATTGGTATGGATGGTTTCAAATCTTTGAGACCACTGGTTTTATCTCTTACATCTCCAAAGGGCTTG
CAAAAGCTACAAAAAAATCGTAGAAATCTGGCAGAAAGAATAATCGAAGTTGTAAATAACTTTGGAAGCG
AATTATTCCCCGATTTCGATAGTGCCGCCCTACGTGAAGAATTCGACGTCATCGATGAAGAGGAAGAAGA
TCGAAAAACCTCAGAATTGAATCGCAGGAAAATGCTAAGAAAACAGAAAATAAAAAGACAAGAAAAAGAT
TCGTCATCACCTATCATCAGCCAACGTGACAACCACGATGCCTATGAACACCATAACCAAGATTCCGATG
GCGTCTCATTGGTCAATAGTGACAATTCCCTCTCTAACATTCCATTATTCTCTTCTACTTTTCATCGTAA
GTCAGAGTCTTCCTTAGCTTCGACATCCGTTGCACCTTCTTCTTCCTCCGAATTTGAGGTAGAAAACGAA
ATCTTGGAGGAAAAAAATGGATTAGCAAGTAAAATCGCACAGGCCGTCTTAAACAAGAGAATTGGTGAAA
ATACTGCCAGGGAAGAGGAAGAGGAAGAAGAAGAGGAAGAAGAAGAAGAGGAAGAAGAAGAAGAAGGGAA
AGAAGGAGATGCGTAG-3′.

### BRIEF DESCRIPTIONS OF THE DRAWINGS

FIG. 1. Conserved motifs of YBL011w and YKR067w encoding proteins in comparison to known glycerol-3-phosphate acyltransferase sequences. (A) Alignment of YBL011w encoded protein sequence [SEQ ID NO:15] and YKR067w [SEQ ID NO: 16] encoded protein sequence with partial sequences of G-3-P acyltransferase from Escherichia coli (P1sB; accession no. P00482) [SEQ ID NO:13] and Rattus norvegicus (RGPAT; accession no. NP_{—}058970) [SEQ ID NO:14], using MegAlign® program from the software package DNAstar. Identical amino acid residues are highlighted in shade. The glycine residue in protein encoded by YKR067w, which is converted to an aspartic acid as a result of a point mutation in TTA1, is marked with an asterisk. (B) Hydropathy profiles of the acyltransferases predicted with the Kyte-Doolittle algorithm. An average of 9 residues is plotted for hydropathy value. Hydrophilic regions are defined as positive values, and hydrophobic regions as negative values. The abscissa is the residue number at the center of each stretch.

FIG. 2. G-3-P acyltransferase activity in strain BY4742 (WT), YKR067w and YBL011w gene disruption strains. Cells of gene disruption strain YKR067w::kanMX4 (A) and YBL011w::kanMX4 (B), as well as the parental strain BY4742 (C) were grown in YPD medium to a late logarithmic phase and used to measure acyltransferase activity in total homogenate preparations.

FIG. 3. G-3-P acyltransferase activities in E. coli strain BB26-36 expressing the wild-type (Gat1) and mutant forms (Gat1m) of YKR067w gene. BB26-36 cells harboring Gat1 and Gat1m expression vectors were cultured and G-3-P acyltransferase activity was measured. Background enzyme activity in the cells bearing the control vector pQE60 was also shown.

FIG. 4. G-3-P and DHAP acyltransferase activities in the ΔGat1 strain over-expressing Gat1 and Gat2 genes. Expression vector pYES2 harboring Gat1 and Gat2, respectively, were introduced into the ΔGat1 yeast strain, and assay of the G-3-P acyltransferase (GAT) and DHAP acyltransferase (DHAPAT) activities.

FIG. 5. Fatty acyl substrate specificity of the Gat1p and Gat2p. Gat1 and Gat2 were inserted into yeast expression vector pYES2, and expressed in the ΔGat1 strain. G-3-P acyltransferase activity from cells containing vector alone was used as a control. Fatty acyl substrates used in the assays were palmitoyl -CoA (16:0-CoA), palmitoleoyl-CoA (16:1-CoA), stearoyl-CoA (18:0-CoA), and oleoyl-CoA (18:1-CoA).

FIG. 6. Relative phospholipid compositions of ΔGat1and ΔGat2 and the wild-type strain BY4742. Wild type, Gat1 and Gat2 deletion strains grown in YPD medium to a late logarithmic phase were used for lipid extraction. The abbreviations used are: PC, phosphatidylcholine; PE, phosphatidylethanolamine; PS, phosphatidylserine; PI, phosphatidylinositol; PA, phosphatidic acid, DMPE, dimethylphosphatidylethanolamine.

### TERMINOLOGY

In the context of this disclosure, a number of terms are utilized. These terms are briefly described below.

As used herein, a “polynucleotide” is a polymer of RNA or DNA that is single- or double-stranded, optionally containing synthetic, non-natural or altered nucleotide bases. A polynucleotide in the form of a polymer of DNA may be comprised of one or more segments of cDNA, genomic DNA or synthetic DNA.

As used herein, “substantially similar” refers to nucleic acid fragments wherein changes in one or more nucleotide bases results in substitution of one or more amino acids, but do not affect the functional properties of the polypeptide encoded by the nucleotide sequence. “Substantially similar” also refers to nucleic acid fragments wherein changes in one or more nucleotide bases does not affect the ability of the nucleic acid fragment to mediate alteration of gene expression by gene silencing through for example antisense or co-suppression technology. “Substantially similar” also refers to modifications of the nucleic acid fragments of the present invention such as deletion or insertion of one or more nucleotides that do not substantially affect the functional properties of the resulting transcript vis-a-vis the ability to mediate gene silencing or alteration of the functional properties of the resulting protein molecule. It is therefore understood that the invention encompasses more than the specific exemplary nucleotide or amino acid sequences and includes functional equivalents thereof.

For example, it is well known in the art that antisense suppression and co-suppression of gene expression may be accomplished using nucleic acid fragments representing less than the entire coding region of a gene, and by nucleic acid fragments that do not share 100% sequence identity with the gene to be suppressed. Moreover, alterations in a nucleic acid fragment which result in the production of a chemically equivalent amino acid at a given site, but do not effect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

Moreover, substantially similar nucleic acid fragments may also be characterized by their ability to hybridize. Estimates of such homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C.

Substantially similar nucleic acid fragments of the present invention may also be characterized by the percent identity of the amino acid sequences that they encode to the amino acid sequences disclosed herein, as determined by algorithms commonly employed by those skilled in this art. Preferred are those nucleic acid fragments whose nucleotide sequences encode amino acid sequences that are 80% identical to the amino acid sequences reported herein. More preferred nucleic acid fragments encode amino acid sequences that are 90% identical to the amino acid sequences reported herein. Most preferred are nucleic acid fragments that encode amino acid sequences that are 95% identical to the amino acid sequences reported herein. Sequence alignments and percent identity calculations were performed using the Megalign program of the LASARGENE bioinformatics computing suite (DNASTAR Inc., Madison, Wis., U.S.A.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

A “substantial portion” of an amino acid or nucleotide sequence comprises an amino acid or a nucleotide sequence that is sufficient to afford putative identification of the protein or gene that the amino acid or nucleotide sequence comprises. Amino acid and nucleotide sequences can be evaluated either manually by one skilled in the art, or by using computer-based sequence comparison and identification tools that employ algorithms such as BLAST (Basic Local Alignment Search Tool; Altschul et al. (1993) J. Mol. Biol. 215:403-410; see also www.ncbi.nlm.nih.gov/BLASTO/). In general, a sequence of ten or more contiguous amino acids or thirty or more contiguous nucleotides is necessary in order to putatively identify a polypeptide or nucleic acid sequence as homologous to a known protein or gene. Moreover, with respect to nucleotide sequences, gene-specific oligonucleotide probes comprising 30 or more contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12 or more nucleotides may be used as amplification primers in PCR in order to obtain a particular nucleic acid figment comprising the primers. Accordingly, a “substantial portion” of a nucleotide sequence comprises a nucleotide sequence that will afford specific identification and/or isolation of a nucleic acid fragment comprising the sequence. The present specification teaches amino acid and nucleotide sequences encoding polypeptides that comprise one or more particular plant proteins. The person skilled in the art, having the benefit of the sequences as reported herein, may now use all or a substantial portion of the disclosed sequences for purposes known to those skilled in this art. Accordingly, the present invention comprises the complete sequences as reported in the accompanying Sequence Listing, as well as substantial portions of those sequences as defined above.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a maimer different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a nucleotide sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions. Promoters which cause a nucleic acid fragment to be expressed in most cell types at most times are commonly referred to as “constitutive promoters”. New promoters of various types useful in plant cells are constantly being discovered; numerous examples may be found in the compilation by Okamuro and Goldberg (1989) Biochemistry of Plants 15:1-82. It is further recognized that since in most cases the exact boundaries of regulatory sequences have not been completely defined, nucleic acid fragments of different lengths may have identical promoter activity.

The term “operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020, incorporated herein by reference).

“Altered levels” refers to the production of gene product(s) in transgenic organisms in amounts or proportions that differ from that of normal or non-transformed organisms. Altered levels are average values of a significant number of transgenic organisms that differ measurably from equivalent average values of non-transformed organisms of the same kind produced under the same conditions at the same time.

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050, incorporated herein by reference).

Standard recombinant DNA and molecular cloning techniques used herein are well known in the art and are described more fully in Sambrook et al., Molecular Cloning: A Laboratory Manual; Cold Spring Harbor Laboratory Press: Cold Spring Harbor, 1989.

### BEST MODES FOR CARRYING OUT THE INVENTION

The present invention describes nucleic acids encoding novel G-3-P/DHAP acyltransferase activities. These two enzymatic activities represent the two key fatty acyltransferases of the glycerolipid biosynthesis pathway in Saccharomyces cerevisiae. In the present invention, mutants of yeast that have altered lipid biosynthesis were analyzed for the molecular nature of the mutation. As a result of this analysis, the coding regions of two acyltransferases with specificity towards G-3-P and DHAP were identified [SEQ ID NOS:5 and 7]. The amino acid sequence [SEQ ID NOS: 6 and 8] and the gene sequencing encoding these two enzymatic activities were previously not known. These enzymes represent the cytoplasmic forms of G-3-P/DHAP acyltransferase, one enzyme has equal affinity for G-3-P and DHAP as acceptors for an acyl group, while the other has a higher affinity for G-3-P over DHAP as an acceptor for an acyl group.

In the following discussion, it will be appreciated that references to the specific novel sequences described herein is intended to include references to substantially similar sequences and substantial portions of such sequences.

In one aspect of the invention these nucleic acid sequences may be used for identification of related homologous sequences deposited in public databases through comparative techniques well-known in the art, or as a hybridization probe for the identification of related cDNA or genomic sequences from various species, including plant species where the DNA sequence information is not known. As noted, isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g. polymerase chain reaction, ligase chain reaction, etc.). In particular, it is contemplated that these sequences so described can be used for the isolation of plant genes encoding the same enzymatic activities.

For example, genes encoding other G-3-P and DHAP acyltransferase genes, either as cDNAs or genomic DNAs, may be isolated directly by using all or a portion of the present nucleic acid fragments as DNA hybridization probes to screen libraries from any desired plant employing methodology well known to those skilled in the art. Specific oligonucleotide probes based upon the present nucleic acid sequences can be designed and synthesized by methods known in the art (Maniatis, T., Frittsch, E. F., and Sambrook, J. (1982; Molecular Cloning: A Laboratory Manual, Cold Spring Harbor, N.Y.). Moreover, the entire sequences can be used directly to synthesize DNA probes by methods known to the person skilled in the art such as random primer DNA labeling, nick translation, or end-labeling techniques, or RNA probes using available in vitro transcription systems. In addition, specific primers can be designed and used to amplify a part or all of the present sequences. The resulting amplification products can be labeled directly during amplification reactions or labeled after amplification reactions, and used as probes to isolate full length cDNA or genomic fragments under conditions of appropriate stringency.

In addition, two short segments of the present nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the present nucleic acid fragments, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, the person skilled in the art can follow the RACE protocol (Frohman et al. (1988) Proc. Natl. Acad. Sci. USA 85:8998) to generate cDNAs by using PCR to amplify copies of the region between a single point in the transcript and the 3′ or 5′ end. Primers oriented in the 3′ and 5′ directions can be designed from the present sequences. Using commercially available 3′ RACE or 5′ RACE systems (BRL), specific 3′ or 5′ cDNA fragments can be isolated (Ohara et al. (1989) Proc. Natl. Acad. Sci. USA 86:5673; Loh et al. (1989) Science 243:217). Products generated by the 3′ and 5′ RACE procedures can be combined to generate full-length cDNAs (Frohman and Martin (1989) Techniques 1:165).

The nucleic acid sequences provided in the present invention can be used to alter the lipid composition in yeast cells and can be expressed under different regulatory elements than normally found associated with said sequences. As one object of the present invention, it is contemplated that these genes can be expressed in higher cells to alter lipid biosynthesis.

The nucleic acid sequences encoding said enzymes provided in the present invention can be used to alter the lipid composition in heterologous cells and can be expressed under different regulatory elements optimized for expression in said heterologous cells. As one object of the present invention, it is contemplated that these genes can be expressed in plant cells to alter lipid biosynthesis.

The nucleic acid fragments of the present invention may be used to create transgenic plants in which the disclosed polypeptides are present at higher or lower levels than normal or in cell types or developmental stages in which they are not normally found. This would have the effect of altering the level of acylated glycerol-3-phosphate or acylated di-hydroxyglycerol phosphate. This leads to changes in overall lipid content or composition, in particular altered levels of lipids in the seed of plants capable or storing lipids in the seed.

Overexpression of the proteins of the present invention may be accomplished by first constructing a chimeric gene in which the coding region is operably linked to a promoter capable of directing expression of a gene in the desired tissues at the desired stage of development. For reasons of convenience, the chimeric gene may comprise promoter sequences and translation leader sequences derived from the same genes. 3′ Non-coding sequences encoding transcription termination signals may also be provided. The present chimeric gene may also comprise one or more introns in order to facilitate gene expression.

Plasmid vectors comprising the present chimeric gene can then constructed. The choice of plasmid vector is dependent upon the method that will be used to transform host plants. The person skilled in the art is well aware of the genetic elements that must be present on the plasmid vector in order to successfully transform, select and propagate host cells containing the chimeric gene. The person skilled in the art will also recognize that different independent transformation events will result in different levels and patterns of expression (Jones et al. (1985) EMBO J. 4:2411-2418; De Almeida et al. (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

For some applications it may be useful to direct the present polypeptides to different cellular compartments, or to facilitate its secretion from the cell. It is thus envisioned that the chimeric gene described above may be further supplemented by altering the coding sequence to encode the present polypeptides with appropriate intracellular targeting sequences such as transit sequences (Keegstra (1989) Cell 56:247-253), signal sequences or sequences encoding endoplasmic reticulum localization (Chrispeels (1991) Ann. Rev. Plant Phys. Plant Mol. Biol. 42:21-53), or nuclear localization signals (Raikhel (1992) Plant Phys. 100: 1627-1632) added and/or with targeting sequences that are already present removed. While the references cited give examples of each of these, the list is not exhaustive and more targeting signals of utility may be discovered in the future.

It may also be desirable to reduce or eliminate expression of genes encoding the present polypeptides in plants for some applications. In order to accomplish this, a chimeric gene designed for co-suppression of the present polypeptide can be constructed by linking a gene or gene fragment encoding that polypeptide to plant promoter sequences. Alternatively, a chimeric gene designed to express antisense RNA for all or part of the present nucleic acid fragment can be constructed by linking the gene or gene fragment in reverse orientation to plant promoter sequences. Either the co-suppression or antisense chimeric genes could be introduced into plants via transformation wherein expression of the corresponding endogenous genes are reduced or eliminated. It may be desirable to first isolate the corresponding G-3-P/DHAP sequence from the plant species in question to ensure that homology based down-regulation of gene activity is carried out using a sequence that is highly homologous to the expressed sequence, or use a portion of the exact sequence that is expressed in order to ensure high levels of down-regulation of gene expression.

Molecular genetic solutions to the generation of plants with altered gene expression have a decided advantage over more traditional plant breeding approaches. Changes in plant phenotypes can be produced by specifically inhibiting expression of one or more genes by antisense inhibition or cosuppression (U.S. Pat. Nos. 5,190,931, 5,107,065 and 5,283,323). An antisense or cosuppression construct would act as a dominant negative regulator of gene activity. While conventional mutations can yield negative regulation of gene activity these effects are most likely recessive. The dominant negative regulation available with a transgenic approach may be advantageous from a breeding perspective. In addition, the ability to restrict the expression of specific phenotype to the reproductive tissues of the plant by the use of tissue specific promoters may confer agronomic advantages relative to conventional mutations which may have an effect in all tissues in which a mutant gene is ordinarily expressed.

The person skilled in the art will know that special considerations are associated with the use of antisense or cosuppresion technologies in order to reduce expression of particular genes. For example, the proper level of expression of sense or antisense genes may require the use of different chimeric genes utilizing different regulatory elements known to the person skilled in the art. Once transgenic plants are obtained by one of the methods described above, it will be necessary to screen individual transgenics for those that most effectively display the desired phenotype. Accordingly, the person skilled in the art will develop methods for screening large numbers of transformants. The nature of these screens will generally be chosen on practical grounds, and is not an inherent part of the invention. For example, one can screen by looking for changes in gene expression by using antibodies specific for the protein encoded by the gene being suppressed, or one could establish assays that specifically measure enzyme activity. A preferred method will be one which allows large numbers of samples to be processed rapidly, since it will be expected that a large number of transformants will be negative for the desired phenotype.

In one aspect of the present invention, these gene sequences are used to modify lipid composition by the transformation of plant cells with a plant transformation vector comprising a sense portion of the gene encoding a G-3-P or DHAP acyltransferase activity. In the context of the present invention, modification means the alteration of lipid content or composition in one or more plant tissues. This can include reduction or increase in lipid content, reduction or increase in one or more of the lipid components.

In another aspect of the present invention, these gene sequences are used to modify lipid composition by the transformation of plant cells with a plant transformation vector comprising antisense portion of the gene capable of hybridizing to an expressed plant G-3-P or DHAP acyltransferase gene, or a double stranded RNA comprising of both sense and antisense portions of the gene with homology to an expressed plant G-3-P or DHAP acyltransferase gene.

In another aspect of the present invention, these gene sequences are used to modify lipid composition by the transformation of plant cells with a plant transformation vector comprising a coding region of said gene under the control of a tissue-specific promoter, most preferably a seed specific promoter such that seed with altered lipid content or composition is derived. Examples of seed specific promoters include the napin promoter from Brassica napus, or the Phaseolin promoter from Phaseolus spp.

In another aspect of the invention, methods for the isolation of cytoplasmic forms of G-3-P and DHAP acyltransferases from yeast is described. It is generally known that lipid biosynthesis enzymes can be localized to various cellular fractions such as mitochondria, plastids and the cytoplasm. Lipid biosynthesis enzymes can be localized to membranes or soluble in the cytoplasm, typically in association with a “lipid body”. Said enzymes described herein represent cytoplasmic forms of these enzymes, previously not identified.

In order to isolate the G-3-P and DHAP acyltransferases, mutant strains of yeast were used that exhibited altered lipid profiles and biosynthesis. The nature of these mutants was analyzed and a strategy was devised to discover the molecular nature of these mutations. It is contemplated that a similar strategy of mutant complementation can be used to discover cytoplasmic localized forms of G-3-P and DHAP acyltransferase genes in other organisms. The variations thereof and modification to the described method of identification of the G-3-P and DHAP acyltransferase enzyme will be apparent to those skilled in the art. Accordingly the application of the method is not limited to yeast.

Two mutant yeast strains were used to discover the G-3-P and DHAP acyltransferase genes. The first mutant analyzed was the yeast ise mutant, a conditional choline auxotrophic mutant. Its growth is inhibited by high inositol, but this defect can be suppressed by supplementation of choline to the inositol-containing medium (Yamashita, S., and Oshima, A. (1980) Eur. J. Biochem. 104, 611-616). The growth defect of ise mutant in response to inositol has been shown to be due to a dramatic decrease in the phosphatidylethanolamine (PE). Choline supplementation suppresses the growth defect, but does not reverse the decrease in the enzyme activities of PE methyltransferases imposed by high inositol, indicating that the supply of choline may lead to an increase in phosphatidylcholine (PC) synthesis via the CDP-choline pathway. In ise mutants, upon the suppression of the CDP-DAG pathway by inositol, the activity of choline transporter (CTR1) becomes essential for phospholipid biosynthesis through the CDP-choline pathway (Nikawa, J., Tsukagoshi, Y., and Yamashita, S. (1986) J. Bacteriol. 166, 328-330; Nikawa, J., Hosaka, K., Tsukagoshi, Y., and Yamashita, S. (1990) J. Biol. Chem. 265, 15996-16003).

A second mutant, the choline transporter mutant, ctr1, has a marked decrease in choline supply, and thereby a weakened CDP-choline pathway for PC synthesis. These two mutants can be combined to form a double mutant.

The ise ctr1 double mutant showed a growth defect even in the presence of choline when high levels of inositol is present in the medium (Nikawa, J., Tsukagoshi, Y., and Yamashita, S. (1986) J. Bacteriol. 166, 328-330). This indicates that the combination of a crippled CDP-choline pathway and a PE methylation pathway is the root cause in the growth defect in lipid biosynthesis of the ise ctr 1 double mutant at high levels of inositol. The ise ctr1 double mutant cannot grow on high inositol medium even in the presence of choline supplement. Such a growth defect is apparently caused by a reduced synthesis of phosphatidial choline (PC).

It has been reported that a choline transporter suppressor gene, SCT1, corresponding to ORF YBL011w (also annotated as YBL03.09), when expressed via a multicopy vector, could complement the cell growth defect which resulted from the deficiency of choline transport in ise ctr1 (Matsushita, M., and Nikawa, J. (1995) J. Biochem. 117, 447-45). However, SCT1 cannot bypass the null mutation of ctr1, and over expression of SCT1 did not appear to restore choline transport activity. Thus, the nature of the ORF in YBL011w was previously unknown.

In the present invention, we demonstrate that the choline transporter suppressor, SCT1, encoded by YBL011w as well as a closely related protein encoded by YKR067w are two yeast sn-1 acyltransferases catalyzing both G-3-P and DHAP acylation. This demonstration includes sequence comparison to known acyltransferases, biochemical characterization of mutants and expression of ORFs in heterologous hosts to confirm enzymatic activity and specificity.

These discoveries demonstrate that the gene sequences in the open reading frames found in YKR067w and YBL011w, designated herein as Gat1 and Gat2, respectively, are yeast G-3-P and DHAP acyltransferase genes. The proteins encoded for by these open reading frames are referred to as Gat1p and Gat2p.

In the present invention it has been shown that Gat2p encoded by YBL011w and the closely related Gat1p encoded by YKR067w; are G-3 -P acyltransferases, in part based on the analysis of their sequences in which two regions similar to the conserved motifs of known acyltransferases were discovered.

Sequence analysis of the protein encoded by the ORFs revealed that the protein encoded by YBL011w, and a protein encoded by YKR067w which displays 31% sequence identity, both contained segments with similarities to conserved domains of known acyltransferases. Two short segments of the proteins encoded by YBL011w and YKR067w resemble the conserved motif III and IV, respectively, of G-3-P acyltransferases. The region corresponding to motif III is accentuated by a stretch of 6 amino acids (IFPEGG) highly conserved among not only G-3-P acyltransferases, but also LPA acyltransferases.

The structure similarity between these newly identified yeast proteins and other known membrane based G-3-P acyltransferase can be further inferred by hydropathy profiles of the encoded protein. The combination of the evidence indicates the proteins encoded by YBL011w and YKR067w are sn-1 fatty acyltransferase.

Biochemical results presented confirm that the proteins encoded by the Gat1 and Gat2 genes, (Gat1p and Gat2p) are G-3-P/DHAP dual substrate-specific sn-1 acyltransferases. The fatty acyl specificity of Gat1 p is similar to that of the mammalian microsomal G-3-P acyltransferase as it can effectively utilize a broad range of fatty acids as acyl donors. In contrast, Gat2p displayed preference towards 16-carbon fatty acids.

Additional evidence found was that the disruption of either Gat1 or Gat2 genes resulted in a reduction in the total cellular G-3-P acyltransferase activities. This was further substantiated by a point mutation revealed in Gat1 of the G-3-P acyltransferase mutant TTA1. In addition, over-expression of the Gat1 and Gat2 genes in the ΔGat1 strain, which has a low G-3-P acyltransferase background, led to highly elevated enzyme activities. Finally, expression of Gat1 in E. coli strain BB26-36 demonstrated a direct enzyme-protein relationship. Thus, the present invention has assigned a function and activity to previously unknown open reading frames. These activities are G-3-P/DHAP acyltransferase.

Accordingly, the nucleic acid sequences provided encode two previously uncharacterized enzymes capable of acylation of G-3-P and DHAP.

The identification of this unique genetic activity allows for novel strategies to manipulate lipid pathways and lipid content and composition in cells. In addition to the use of these novel nucleic acid sequences for the genetic modification of lipid content, the sequence can also be used to isolate corresponding related similar or identical sequences from other species, including plant species.

Accordingly, in one embodiment of the invention the subject method includes he steps of expressing a G-3-P/DHAP acyltransferase gene in a heterologous species comprising the steps of:

a) introducing into a cell capable of being transformed a genetic construct comprising a first DNA expression cassette that comprises, in addition to the DNA sequences required for transformation and selection in said cells, a DNA sequence that encodes a G-3-P/DHAP acyltransferase coding sequence, operably linked to a suitable transcriptional regulatory region and,

(b) recovery of a cell which contains said recombinant DNA.

The cell containing the recombinant DNA can exhibit altered lipid content or composition when compared to cells without the recombinant DNA construct. Thus, the method finds utility in the alteration of lipid content in various cells. These alterations can lead to changes in cellular phenotype.

It is known that plant species are used as a source of lipids, typically triglycerides and that alteration of oil composition in plant cells is an important commercial objective. Attempts to increase oil content, alter the oil profile, or change the overall composition of plant lipids have been the subject of research for many decades. In particular the use of heterologous genes for modifying oil composition of plant cells is well established (e.g., Budziszewski et. al., Lipids, 31:557-569, 1996).

Increases in specific content of oils in plants has been accomplished by expression of various heterologous genes capable of acting upon fatty acid substrates. Expression of the medium chain fatty acyl-ACP thioesterase from the California Bay plant has been shown to increase the content of lauric acid in Brassica plant seeds (Voelker et al., Science 257:72-74, 1992, also U.S. Pat. No. 5,455,167, U.S. Pat. No. 5,512,482, U.S. Pat. No. 5,639,790, U.S. Pat. No. 5,654,495, the disclosures of which are incorporated herein by reference).

Modification of seed oil by expression of a yeast derived sn-2 acyltransferase in Brassica has also been demonstrated to alter oil composition and content (Zou et al., The Plant Cell 9:909-923, 1997, the disclosure of which is incorporated herein by reference). This demonstrates the utility in using acyltransferase genes and sequences from various species, including by extension those sequences derived from yeast in modifying oil composition and content in plant seeds.

Additional manipulations of interest include the increase in the addition of long chain fatty acids at a specific position on glycerol backbones. For example the formation of tri-erucic acid in canola and hence an increase in erucic acid production using a heterologous acyltransferase has been described (U.S. Pat. No. 5,563,058, U.S. Pat. No. 5,824,858, U.S. Pat. No. 6,093,568, WO078974, the disclosures of which are incorporated herein by reference). Additional modification of oil content in plant seeds has been demonstrated by expression of a heterologous acyltransferase for example, oil seed rape (Brassica napus) transformed with a 2-acyltransferase transgene derived from Limnanthes douglassi in order to increase the erucic acid content of the oil. A primary objective is the formation of tri-erucic acid varieties.

Accordingly, the utility of genes capable of modification of fatty acids in plant cells has been demonstrated. In particular, heterologous genes have been shown to provide compositional changes, as well as changes in content of fatty acids in plants that have important industrial applications. Genes from heterologous organisms provide many advantages for modifying oil since their DNA sequences are typically not subject to the same regulatory pathway as found for the oil biosynthesis genes normally associated with the plant. In addition the isolated genes can also be placed under the control of novel regulatory elements, providing new genetic combinations for modifying oil, with expression being limited to the seeds or organs that accumulate oil. Thus, it is clear that the current invention provides an additional means to alter oil content in plant seeds through the disclosure of two novel enzymatic activities and the genes encoding these enzymes.

Of particular interest are plants and plant seeds from oilseed crops. Crops grown for oil extraction include both edible and industrial oil crops. For example, edible oil crops can include, but are not limited to canola (Brassica spp.), Soybean (Glycine and Soja spp.), Sunflower (Helianthus spp.), Cotton (Gossypium spp.), Corn (Zea mays), Olive (Olea spp.) Safflower (Carthamus spp.), Cocoa (Theobroma cacoa), Peanut (Arachis spp.), Flax (Linum spp.) as well as crops that have industrial utility, e.g., Castor (Ricinus spp.), rapeseed, high erucic acid Brassica, Lesquerella, Limnanthes and others.

Modification of the oil content and composition of these crops can provide many important benefits including decreased or increased content of specific fatty acids, increased overall oil content to improve the economics of oil production and seed with higher fatty acid content and hence higher energy content for feed applications.

The discovery of a polynucleotide sequence encoding a novel G-3-P/DHAP acyltransferase allows for the modification of plant cells in a manner heretofore unknown. These novel enzymatic activities can be used directly by expression in plant cells under the control of an appropriate plant promoter, or can be used to isolate related plant genes by techniques well known in the art. In particular, the invention contemplates the modification of plant cells by expression of said polynucleotides encoding G-3-P/DHAP acyltransferase activity.

Accordingly, in a preferred embodiment of the invention the subject method includes a method for modifying the lipid composition of a plant cell comprising:

(a) Introducing into a plant cell capable of being transformed and regenerated to a whole plant a genetic construct comprising a first DNA expression cassette that comprises, in addition to the DNA sequences required for transformation and selection in plant cells, a DNA sequence (Seq. I.D. No. 5) that comprises a polynucleotide region encoding a G-3-P/DHAP acyltransferase sequence, operably linked to a suitable transcriptional regulatory region and,

(b) recovery of a plant cell which contains said recombinant DNA and has altered lipid content or composition.

In another preferred embodiment of the invention the subject method includes method for modifying the lipid composition of a plant cell comprising:

(a) Introducing into a plant cell capable of being transformed and regenerated to a whole plant a genetic construct comprising a first DNA expression cassette that comprises, in addition to the DNA sequences required for transformation and selection in plant cells, a DNA sequence (Seq. I.D. No. 6) that comprises a polynucleotide region encoding a G-3-P/DHAP aclytransferase sequence, operably linked to a suitable transcriptional regulatory region and,

(b) recovery of a plant cell which contains said recombinant DNA and has altered lipid content or composition.

The chimeric gene is introduced into a plant cell and a plant cell recovered wherein said gene is integrated into the plant chromosome. The plant cell is induced to regenerate and a whole plant is recovered. The use of these techniques has been well-described in the art, and it is apparent that the entire G-3-P/DHAP polynucleotide sequence, or portions thereof can be employed within the scope of the present invention.

The method further relies on the use of transformation to introduce the gene encoding the enzyme into plant cells. Transformation of the plant cell can be accomplished by a variety of different means. Methods that have general utility include Agrobacterium based systems, using either binary and cointegrate plasmids of both A. tumifaciens and A. rhyzogenies. (e.g., U.S. Pat. No. 4,940,838, U.S. Pat. No. 5,464,763), the biolistic approach (e.g, U.S. Pat. No. 4,945,050, U.S. Pat. No. 5,015,580, U.S. Pat. No. 5,149,655), microinjection, (e.g., U.S. Pat. No. 4,743,548), direct DNA uptake byprotoplasts, (e.g., U.S. Pat. No. 5,231,019, U.S. Pat. No. 5,453,367) or needle-like whiskers (e.g., U.S. Pat. No. 5,302,523). Any method for the introduction of foreign DNA and/or genetic transformation of a plant cell may be used within the context of the present invention.

The method also relies on the recovery and use of the plant cells or tissue with the altered properties, particularly plant tissue with altered lipid content or composition. These tissues can include seed tissue or whole plant tissue or other tissue that would benefit from altered lipid composition.

It is also apparent to one skilled in the art that the polynucleotide and deduced amino acid sequence of the G-3-P/DHAP acyltransferase can be used to isolate related genes from various other species, including plant species. The similarity or identity of two polypeptide or polynucleotide sequences is determined by comparing sequences. In the art, this is typically accomplished by alignment of the amino acid or nucleotide sequences and observing the strings of residues that match. The identity or similarity of sequences can be calculated by known means including, but not limited to, those described in Computational Molecular Biology, Lesk A. M., ed., Oxford University Press, New York, 1988, Biocomputing: Informatics and Genome Projects, Smith, D. W., ed., Academic Press, New York, 1993., Computer Analysis of Sequence Data, Part I, Griffin, A. M. and Griffin, H. G., eds., Humana Press, New Jersey, 1994 and other protocols known to those skilled in the art. Moreover, programs to determine relatedness or identity are codified in publicly available programs. One of the most popular programs comprises a suite of BLAST programs, three designed for nucleic acid sequences, (BLASTN, BLASTX and TBLASTX) and two designed for protein sequences (BLASTP and TBLASTN) (Coulson, Trends in Biotechnology, 12:76-80, 1994). The BLASTX program is publicly available from NCBI and other sources such as the BLAST Manual, Altschul, S., et al., NCBI NLM NIH Bethesda Md. 20984, also http://www.ncbi.nlm.nih.gov/BLAST/blast help.html) provides online help and further literature references for BLAST and related protein analysis methods, and Altschul, S., et al., J. Mol. Biol 215:403-410, 1990.

The isolated polynucleotide can be sequenced and the DNA sequence used to further screen DNA sequence collections to identify related sequences from other species. The DNA sequence collections can comprise EST sequences, genomic sequences or complete cDNA sequences. In particular, similarity at the protein level in areas known to be conserved in G-3-P/DHAP acyltransferases can be used for preliminary identification of homologous proteins.

The identification of a polynucleotide sequence from a plant species encoding a related enzymatic activity allows for other strategies of manipulation of lipid content or composition. The use of gene inhibition technologies such as antisense RNA or cosuppression or double stranded RNA interference is contemplated within the scope of the present invention. In these approaches, the isolated gene sequence is operably linked to a suitable regulatory element.

It is apparent to the person skilled in the art that the polynucleotide encoding the G-3-P/DHAP aclytransferase sequence can be in the antisense (for inhibition by antisense RNA) or sense (for inhibition by co-suppression) orientation, relative to the transcriptional regulatory region, or a combination of sense and antisense RNA to induce double stranded RNA interference (Chuang and Meyerowitz, PNAS 97: 4985-4990, 2000, Smith et al., Nature 407: 319-320, 2000). A transcriptional regulatory region is often referred to as a promoter region and there are numerous promoters that can be used within the scope of the present invention. In addition, the person skilled in the art will readily recognize that the sequence of the inserted recombinant gene must contain regions of sufficient homology to allow for sequence-specific inhibition of gene expression. Accordingly, for some applications, it is preferable to isolate the specific G-3-P/DHAP acyltransferase from the organism in which reduction of activity is the desired objective. In this fashion, the present invention provides a DNA and protein sequence of utility for isolation of said specific G-3-P/DHAP acyltransferase.

It is obvious to the skilled practitioner that any number of tissue-selective promoters may be employed within the scope of the present invention. In particular a seed-selective promoter is used to alter the lipid composition in crops where seed is used for oil extraction. In other crops various tissue-selective promoters may be used dependent upon the portion of the plant where alteration of lipid content or composition is desired.

The following examples serve to illustrate the method and in no way limit the utility of the invention.

### EXAMPLES

### Example 1

Yeast Strains and Culture Conditions Used for Isolation of G-3/DHAP acyltransferase genes.

The gene disruption strains YBL011w::kanMX4 (ΔGat2) (BY4742, Matα, his3X1, leu2X0, lys2X0, ura3X0, YBL011w::kanMX4), YKR067w::kanMX4 (ΔGat1) (BY4742, Matα, his3X1, leu2X0, lys2X0, ura3X0, YKR067w::kanMX4), and the wild-type strains BY4742 (Matα, his3X1, leu2X0, lys2X0, ura3X0) and DBY746 (Matα, his3-X1, leu2-3, leu2-112, ura3-52, trp1-289) were purchased from Euroscarf. The TTA1 mutant (Matα, his3-X1, leu2-3, leu2-112, ura3-52, trp1-289) was kindly provided by Dr. Robert M. Bell. Cells were cultured at 30° C. in YPD medium containing 1% Bacto-yeast extract, 2% Bacto-peptone, and 2% glucose (Sigma).

### Example 2

Sequence analysis of YBL011w and YKR067w in TTA1 and DBY746.

Genomic DNA (150 ng) from TTA1 and its parental strain DBY746 was used, respectively, to amplify the coding regions of YBL011w and YKR067w genes. PCR amplification was performed in a 50 μl PCR reaction containing 0.2 mM dNTPs, 0.2 μM primers, and 2.5 units pfu DNA polymerase (Stratagene, San Diego, Calif., USA). The primers used for the amplification of YBL011w and YKR067w were:

| | |
|---|---|
| Seq ID. No.1: | 5′-ATGCCTGCACCAAAACTCACGGAG-3′ |
| and | |
| Seq.ID. No.2 | 5′-CTACGCATCTCCTTCTTTCCCTTC-3′ |
| and | |
| Seq. ID. No.3: | 5′-ATGTCTGCTCCCGCTGCCGATCAT-3′ |
| and | |
| Seq. ID. No 4: | 5′-TCATTCTTTCTTTTCGTGTTCTCT-3′ |

respectively.

The PCR program employed was as follows: initial dwell time of 2 min at 94° C., then 32 cycles of denaturation at 94° C. for 30 s, annealing at 60° C. for 30 s and extension at 72° C. for 3 min, followed by extension at 72° C. for 7 min. The amplified DNA fragments were cloned into pCR2.1-TOPO vector (Invitrogen) following the addition of a single 3′ deoxyadenosine through Taq DNA polymerase treatment, and fully sequenced using automated DNA sequencer (Applied Biosystems 373).

The DNA sequence of the ORF in YBL011w (Seq I.D. No. 5) and the DNA sequence of the ORF in YKR067w (Seq. I.D. No. 6) were determined.

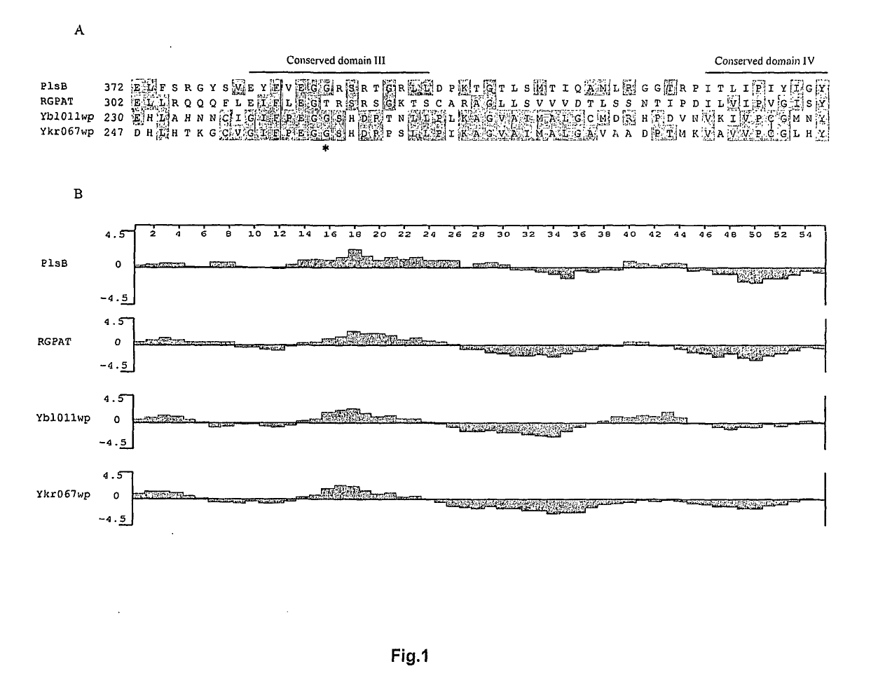

Sequence analysis of the protein encoded by the ORFs revealed that the protein encoded by YBL011w, and a protein encoded by YKR067w which displays 31% sequence identity, both contained segments with similarities to conserved domains of known acyltransferases. A portion of their deduced amino acid sequences are aligned with those of the membrane-bound G-3-P acyltransferases from Escherichia coli and the mitochondrial G-3-P acyltransferase from Rattus norvegicus as shown in FIG. 1A. Two short segments of the proteins encoded by YBL011w and YKR067w resemble the conserved motif III and IV, respectively, of G-3-P acyltransferases. The region corresponding to motif III is accentuated by a stretch of 6 amino acids (IFPEGG) highly conserved among not only G-3-P acyltransferases, but also LPA acyltransferases. The structure similarity between the yeast proteins and other known membrane based G-3-P acyltransferase can be further inferred by hydropathy plot as shown in FIG. 1B. Based on this analyses, it was concluded that the proteins encoded by YBL011w and YKR067w are sn-1 fatty acyltransferase.

### Example 3

Disruption of the genes encoded by YBL011w and YKR067w to reduce G-3-P Acyltransferase Activity

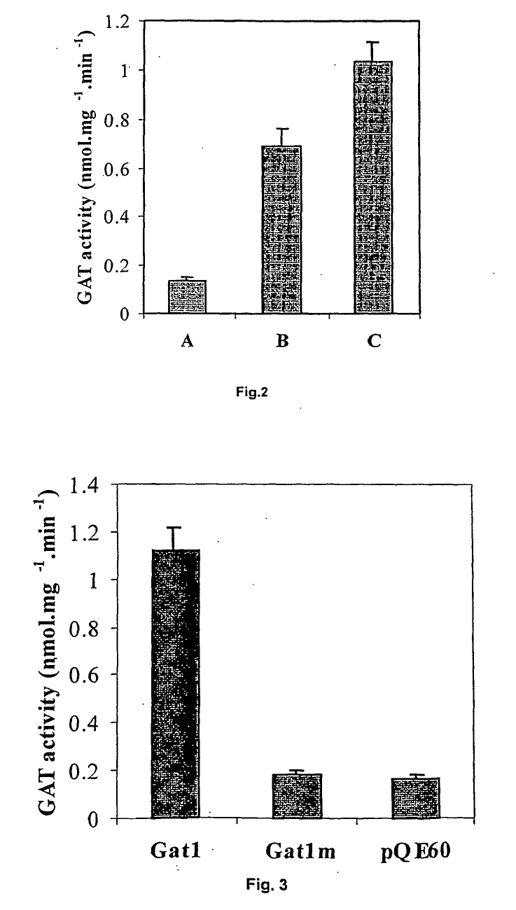

Haploid strains with targeted disruption in YBL011w (EUROSCARF accession no. Y13037) and YKR067W(EUROSCARF accession no. Y15983) were acquired from the collection of deletion strains at EUROSCARF. Neither strain displayed any abnormal growth phenotype when examined on solid or in liquid media. Nor was any apparent sensitivity to temperature or inositol found upon the disruption of the respective open reading frames. Previously, it was shown that a dramatic reduction of G-3-P acyltransferase specific activity could be easily detected even a total homogenate of the yeast strain TTA1 was directly used for enzyme assays. The effect of the gene disruptions on G-3-P acyltransferase activities was conducted by employing total yeast homogenate prepared after a brief spin at 2500 g. As shown in FIG. 2, G-3-P acyltransferase activity was clearly reduced in both gene disruption strains. Disruption of YBL011w reduced G-3-P acyltransferase activity to one third of the wild type level. Disruption of YKR067w, on the other hand, had a more striking effect, leaving a residue enzyme activity at about one eighth of the control (FIG. 2). Under these assay conditions, the level of residual enzyme activity in this YKR067w gene disruption strain is very close to the G-3-P acyltransferase activity for strain TTA1, even though they are derived from somewhat different genetic background.

### Example 4

Identification of a Missense Mutation in the TTA1 Mutant

To further provide evidence for the nature of the activity of the genes encoded by YBL011w and YKR067w in the mutant strain TTA1, the G-3-P acyltransferase and DHAP acyltransferase activities in the mutant TTA1 was examined for comparison to the sequences in YBL011w or YKR067w. The coding regions of the two genes were amplified by Pfu DNA polymerase-based PCR using genomic DNA isolated from TTA1 and its parental strain DBY746. Several nucleotide polymorphic differences were found in both genes between S. cerevisiae strains, and the sequences of YBL011w and YKR067w from DBY746 were deposited into the EMBL database under the accession of AJ314608 and AJ311354, respectively. Both direct sequencing of the purified PCR products and subsequent sequencing of the PCR fragment cloned into a vector plasmid demonstrated that there was no nucleotide sequence change in YBL011w between TTA1 and DBY746. On the other hand, analysis of YKR067w revealed the presence of one nucleotide change from G to A at position 785 in mutant TTA1, which is predicted to result in an aspartic acid to glycine substitution at amino acid position 262 of the encoded protein. Significantly, this amino acid substitution occurred in the segment exhibiting high similarity to the conserved motif III of acyltransferases. This result indicates that the deficiency of acyltransferase activity in TTA1 is attributed to this missense mutation, and thereby suggesting that YKR067w encodes for a G-3-P acyltransferase. The residual G-3-P acyltransferase activity in TTA1 is comparable to that of the YKR067w knockout strain, suggesting that the mutation occurred in TTA1, although in the form of a single amino acid change, completely abolishes the activity of this enzyme. This result is in good agreement with previous observations with the E. coli G-3-P acyltransferase that a change of amino acid sequence in the conserved domain III from YFVEGGRSRTGR to YFVELGRSRTGR completely eliminated its enzyme activity. Our results further support the functional importance of these conserved sequence domains in fatty acyltransferases.

Previously it has been demonstrated that the defect in TTA1affected mainly the acyltransferase activities of the lipid particle preparations. Thus, it can be inferred that YKR067wp is the lipid particle G-3-P acyltransferase. In accordance with the nomenclature proposed by Athenstaedt and Daum (ibid), we named YKR067wp as Gat1p. The protein encoded by YBL011w, which has structural properties of a membrane protein, should be localized in other cytoplasmic membrane compartments. Therefore we designated the protein as Gat2p. The genes corresponding to YKR067w and YBL011w were named Gat1 and Gat2, respectively.

### Example 5

Construction of Expression Vectors

In this example, Gat1 and Gat2 coding sequences were isolated and placed into expression vectors. Two pairs of primers,

Seq. I.D. No. 9:
5′-GGATCCAACATGTCTGCTCCCGCTGCCGATCAT-3′
and
Seq. I.D. No.10:
5′-CTCGAGTCATTCTTTCTTTTCGTGTTCTCT-3′
for Gat1 and Gat1m (Gat1 allele from TTA1),
and
Seq. I.D. No. 11:
5′-GGATCCAACATGCCTGCACCAAAACTCACGGAG-3′
and
Seq. I.D. No. 12:
5′-CTCGAGCTACGCATCTCCTTCTTTCCCTTC-3′

for Gat2 gene, were designed to include BamH I and Xho I restriction sites (underlined). The amplified DNA fragments were first cloned into vector pCR2.1-TOPO (Invitrogen). The orientation of the insert was determined by restriction enzyme digestion. Plasmids containing Gat1, Gat1m, and Gat2 were designated as Gat1/pCR2.1-TOPO, Gat1m/pCR2.1-TOPO and Gat2/pCR2.1-TOPO, respectively. To construct bacterial expression vectors, the coding regions of Gat1, Gat1m and Gat2 were recovered by digestion of Gat1/pCR2.1-TOPO, Gat1m/pCR2.1-TOPO and Gat2/pCR2.1-TOPO with BamH I. Purified DNA fragments were inserted into pQE60 and then transformed into E.coli DH₅α. Prior to transforming the resulting plasmids Gat1/pQE60, Gat1m/pQE60 and Gat2/pQE60 into BB26-36, correct orientation and in-frame fusion of the inserts were confirmed by sequencing.

To construct yeast expression vectors, coding regions of Gat1 and Gat2 genes were excised from Gat1/pCR2.1-TOPO and Gat2/pCR2.1-TOPO through digestion with BamH I and Xho I and inserted into vector pYES2 (Invitrogen). The integrity of the constructs, Gat1/pYES2 and Gat2/pYES2, was verified by sequencing. Transformation of pYES2 and the recombinant pYES2 plasmids into Gat1 deletion strain was performed using lithium acetate according to the standard protocol (Ausubel, F. M., Brent, R., Kingston, R. E., Moore, D. D., Seidman, J. G., Smith, J. A., and Struhl, K. (1994) Current Protocols in Molecular Biology. John Wiley & Sons, Inc. p13.0.1-13.13.9).

### Example 6

Heterologous Expression of Gat1 and Gat2 in E. coli

In this example, the Gat1 and Gat2 genes were expressed in the heterologous host E. coli. The Gat1 and Gat2 as well as the mutant Gat1 allele (Gat1m) from TTA1 were inserted into expression vector pQE60 (Qiagen), and introduced into E. coli (plsB) strain BB26-36 (Bell, R. M. (1974) J. Bacteriol. 117, 1065-1076). BB26-36 has a mutation in plsB that gives rise to a G-3-P acyltransferase with altered properties, in particularly, a lower specific activity. Single colonies containing plasmids Gat1/pQE60, Gat1m/pQE60 and Gat2/pQE60, were cultured in 2 ml LB medium supplemented with 0.4% glucose, 0.1% glycerol and 60 μg/ml ampicillin. After incubation at 37° C. for 6 hr, the cultures were transferred to 50 ml of fresh medium, and allowed to grow until cell density reached OD₆₀₀=0.1 IPTG was then added to a final concentration of 0.1 mM, and the cells were grown at 28° C. for an additional 12 hr to induce protein expression. Cells were harvested by centrifugation at 5000 g for 5 min, washed with 50 mM Tris-HCl (pH 7.5) and resuspended in lysis buffer (50 MM This-HCl, pH8.0, 1 mM EDTA, 1 mM DTT, 10% glycerol). After treatment with 100 μg/ml lysozyme (Sigma) for 30 min on ice, the suspension was sonicated six times on ice with a 15-second burst. The lysate was spun at 2000 g for 5 min to pellet cell debris, and the supernatant was used for enzyme assays. G-3-P acyltransferase activity was assayed at room temperature for 10 min in a 200 μl reaction mixture containing 400 μM [¹⁴C] glycerol 3-phosphate (5550 dpm/nmol), 45 μM palmitoyl-CoA, 75 mM Tris-HCl (pH 7.5), 1 mM DTT, and 2 mM MgCl₂. The reaction mixture was extracted with 3 ml of chloroform-methanol (1:2, v/v) in the presence of 600 μl of 1% HClO₄. After a repeated extraction with another 1 ml chloroform and 1 ml 1% HClO₄, the lower phase of the Bligh-Dyer extract (Bligh, E. G., and Dyer, W. J. (1959) Can. J. Biochem. Physiol. 37, 911-917) was washed three times with 2 ml 1% HClO₄. An aliquot of the chloroform phase was dried under nitrogen, and subject to scintillation counting for radioactivity. Results shown are the means ±S.E. from at least three independent assays. To confirm the reaction products, the chloroform lipid extracts were separated through TLC in a solvent system of chloroform/methanol/acetic acid/5% aqueous sodium bisulfite (100:40:12:4). The R_{f} values for LPA and PA were 0.33 and 0.90, respectively (Hajra, A. K., and Burke, C. (1978) J. Neurochem. 31, 125-134). DHAP acyltransferase activity was measured essentially as described (Bates, E. J., and Saggerson, E. D. (1979) Biochem. J. 182, 751-762) with minor modifications. The reaction was terminated by the addition of 0.8 ml 1% HClO₄, followed by extraction with 3 ml chloroform-methanol (1:2, v/v) and 1 ml chloroform. The lower phase of the Bligh-Dyer extract was washed three times with 2 ml 1% HClO₄, and the radioactivity measured through scintillation counting. The product was also subjected to TLC analysis. The R_{f} value of 1-acyl-DHAP in this system was 0.20. Expression of the proteins was confirmed through SDS-PAGE.

As shown in FIG. 3, G-3-P acyltransferase specific activity in this plsB mutant expressing Gat1 was more than six times higher than that of the control. In contrast, expression of Gat1m,, the TTA1 mutation allele of Gat1, in the plsB mutant showed no enzyme activities beyond the control. In addition, E. coli strain BB26-36 has a G-3-P auxotrophic phenotype as a result of a marked increase in the apparent Kₘ of the G-3-P acyltransferase for G-3-P. Expression of Gat1 using both pQE60 and pET28a vectors in the strain BB26-36, however, failed to complement this defect. In addition, expression of Gat2 gene appeared to be extremely deleterious to the host cells. The growth of the cells expressing this gene was slower by a factor of two when compared to the cells harboring the control vector

### Example 7

Expression of Gat1 and Gat2 in yeast

To over-express Gat1 and Gat2 in yeast, single colonies carrying pYES2 (plasmid-only control) or Gat1/pYES2 and Gat2/pYES2 were inoculated in 10 ml SD-uracil medium with 2% glucose. After incubation at 30° C. for 30 hr, the cells were harvest through spinning at 1500 g for 5 min, and then resuspended in SD-uracil medium with 1% raffinose and 2% galactose (SD induction medium). The cells were then diluted into 50 ml of SD induction medium to obtain a cell density of OD₆₀₀=0.6. After incubation at 30° C. for 7 h to induce the protein expression, the cells were harvested by centrifugation at 1500 g for 5 min at 25° C. For preparation of the yeast homogenates, the cell pellets were washed with 10 volumes of distilled H₂O, and then immediately frozen in liquid nitrogen and stored at −80° C. until use. Yeast homogenates were prepared with glass beads according to standard method (see example 5). Yeast lysate in buffer (50 mM This-HCl, pH8.0, 1 mM EDTA, 1 mM DTT, 10% glycerol) was spun at 2500 g, 4° C. for 5 min to pellet large cell debris, and supernatant was used directly for enzyme assays. For conduction lipid analysis, yeast cell cultures at late logarithmic phase were disrupted with glass beads. Total lipids were extracted according to standard techniques (Folch, J. M., Lees, M. and Sloane-Stanley, G. H. (1957) J. Biol. Chem. 226, 497-509). Separation of phospholipids were performed with two-dimensional TLC on Silica Gel-60 plates and developed in solvent systems as described (Morash, S. C., MacMaster, C. R., Hjelmstad, R. H., and Bell, R. M. (1994) J. Biol. Chem. 269, 28769-28776). Phospholipids were visualized with iodine vapor through a nitrogen stream, scraped off the TLC plates, and transmethylated directly with methanolic-HCl. Fatty acid methyl esters derived from each of the lipid species were analyzed and quantified by gas chromatography. From these data the mole percentages of the analyzed phospholipids was calculated for each lipid. Protein concentration was determined using Bio-Rad Dc protein assay regents (BIO-RAD) and bovine serum albumin as a standard.

### Example 8

Demonstration of the Substrate Specificity of the Gat1p and Gat2p proteins with G-3-P/DHAP Dual Substrate Specific Acyltransferase

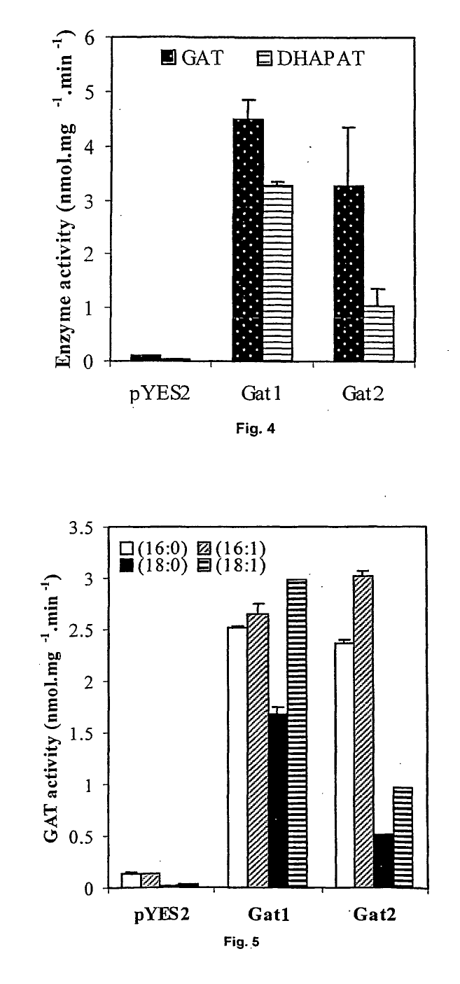

Due to the apparent difficulties involved in the reconstitution of enzyme activities of membrane-bound acyltransferases, we decided to adapt a strategy based on the low G-3-P acyltransferase background of the ΔGat1 strain to investigate the substrate specificities of Gat1p and Gat2p with respect to G-3-P and DHAP. The two genes were expressed using a multiple copy vector pYES2 under the control of GAL⁺ promoter. Specific activities of G-3-P and DHAP acyltransferase of the two enzymes were evaluated using palmitoyl CoA as the fatty acyl donor (FIG. 4). Over-expression of Gat1 resulted in a net increase of 4.39 and 3.17 nmol.mg⁻¹.min⁻¹ in G-3-P acyltransferase and DHAP acyltransferase activities, respectively. Likewise, over-expression of Gat2 led to increases in both enzyme activities (a difference of 3.23 and 0.98 nmol.mg⁻¹.min⁻¹, respectively) (FIG. 4). The observed increases in the specific activities of G-3-P and DHAP acyltransferases indicate that Gat1p and Gat2p can efficiently utilize both G-3-P and DHAP as substrates, thereby providing direct evidence that the two yeast sn-1 acyltransferses are G-3-P/DHAP dual substrate acyltransferases. It is also consistent with the view that Ayr1P, a major component of lipid particles which functions as a 1-acyl-DHAP reductase, works coordinately with Gat1p to carry out the DHAP dependent glycerolipid pathway in yeast lipid particles. As shown in FIG. 4, GAT1p displayed almost the same level of enzyme specific activities with regard to G-3-P and DHAP, while Gat2p clearly preferred G-3-P even though DHAP was also an efficient substrate.

### Example 9

Demonstration of Substrate Specificity of the GAT1p and Gat2p for Specific Fatty Acyl Preferences

Fatty acid substrate specificity of acyltransferases plays important role in determining stereospecific distributions of fatty acyl groups in glycerolipids. Substrate preference in relation to saturated and unsaturated fatty acids has also been frequently implicated in regulations of temperature-dependent incorporation of fatty acids into phospholipids. To investigate the fatty acyl substrate preferences of GAT1p and Gat2p, specific activities towards palmitoyl (16:0)-CoA, palmitoleoyl (16:l)-CoA, stearoyl (18:0)-CoA, and oleoyl (18:1)-CoA were compared using the ΔGat1 strain expressing Gat1 and Gat2, respectively. As shown in FIG. 5, GAT1p could efficiently utilize all four fatty acyl substrates, with a noticeably lower specific activity towards 18:0-CoA. In general, the characteristics of the fatty acyl specificity of Gat1 are similar to that of the mammalian microsomal G-3-P acyltransferase, which is also capable of utilizing a broad range of acyl-CoAs (8). In contrast, Gat2p exhibited considerable preference for 16 carbon fatty acids. Moreover, both enzymes appeared to prefer unsaturated fatty acids to saturated ones.

### Example 10

Demonstration of the Phospholipid and Fatty Acid Profiles of ΔGat1 and ΔGat2 Strains

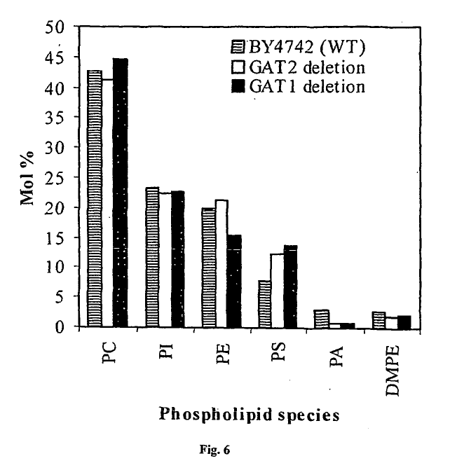

To investigate the respective roles and relative contributions of the two acyltransferases to phospholipid metabolism, the steady-state levels of phospholipids from the ΔGat1 and ΔGat2 strains were compared with those of the parental strain. In accordance with data reported for the TTA1 strain, the size of the phosphatidic acid (PA) pool in the ΔGat1 strain, measured as molar percent of total phospholipids, was reduced to less than half of that of the parental strain (FIG. 6). Similar reduction of the PA pool in the ΔGat2 strain was also observed. This relatively limited PA pool suggests that, in comparison to wild type, the overall flux of glycerolipid synthesis is low due to the disruption of one of the G-3-P acyltransferase genes. Such a decrease implies that sn-1 acyltransferase is a rate-limiting factor in the glycerolipid biosynthetic pathway. Moreover, since a deficiency in either of the isoforms leads to a reduced PA pool, it indicates that Gat1p- and Gat2p-mediated acylations are not entirely redundant as far as maintaining a normal level of phospholipid synthesis flux is concerned. There is also a detectable change in the relative abundance of PS and PI, with a PS/PI molar ratio elevated from 0.33 in the parental strain to 0.60 and 0.55, respectively, in the ΔGat1 and ΔGat2 strains. In light of the fatty acyl substrate specificities of the two acyltransferases, we also examined the fatty acid compositions of the major phospholipid species in ΔGat1 and ΔGat2. The data presented in Table 1 can be summarized as follows: (i) lack of GAT1p in yeast did not seem to have a significant effect on the total fatty acid profiles of PC, PS and PI. However, a decrease in 16:1 fatty acid was observed in PE, and the reduction is proportionally compensated by increases in both 16:0 and 18:1. (ii) the absence of Gat2p impacted fatty acid compositions in all four major phospholipid species. In general, the ΔGat2 mutant had proportionally less 16:0, and such a decrease in 16:0 was offset by increases in other fatty acids, particularly 18:0.

TABLE 1
Fatty acid composition of different
phospholipids in wild-type and mutant strains.

| | | Proportion of fatty acids (Mol %) | | | |
|---|---|---|---|---|---|
| Phospholipid | Strain | 16:0 | 16:1 | 18:0 | 18:1 |
| PC | BY4742 (WT) | 16.06 | 64.17 | 2.76 | 17.0 |
| | YKR067w:kanMX4 | 17.07 | 63.90 | 2.70 | 16.37 |
| | YBL011w:kanMX4 | 10.82 | 66.15 | 4.44 | 18.59 |
| PI | BY4742 (WT) | 43.93 | 19.07 | 12.37 | 24.62 |
| | YKR067w:kanMX4 | 43.69 | 18.83 | 11.86 | 25.62 |
| | YBL011w:kanMX4 | 36.36 | 19.96 | 15.07 | 28.61 |
| PS | BY4742 (WT) | 37.90 | 25.73 | ND^{a} | 36.36 |
| | YKR067w:kanMX4 | 38.10 | 26.46 | ND | 35.41 |
| | YBL011w:kanMX4 | 35.65 | 29.84 | ND | 34.51 |
| PE | BY4742(WT) | 20.12 | 48.17 | ND | 31.71 |
| | YKR067w:kanMX4 | 25.18 | 37.0 | ND | 37.83 |
| | YBL011w:kanMX4 | 18.95 | 47.61 | 2.05 | 31.40 |

The cells grown in YPD medium to late logarithmic phase were used for fatty acid profile analysis.
^{a}ND, not detectable.

### Example 11

Genetic transformation of a plant with a gene encoding a G-3-P/DHAP acyltransferase under the control of a constitutive promoter.

In this example, the coding region of the G-3-P/DHAP acyltransferase found in YKR067 was inserted into a plant transformation vector RD400 (Datla, R. S. S., Hammerlindl, J. K., Panchuk, B., Pelcher, L. E., and Keller, W., 1992, Gene 211:383-384) which as been modified to include instead of the NosP-NptII plant selection marker of RD400 a fusion gene between gus and npt (Gus::npt). The Gus-npt has been described previously (Datla, R. S. S., Hammerlindl, J. K., Pelcher, L. E., Crosby, W. L., and G. Selvaraj, 1991, Gene 101: 239-246). The acyltransferase gene was placed under the control of the 35S promoter and the plasmid was used to transform Brassica plants according to standard protocols.

### Example 12

Genetic transformation of a plant with a gene encoding a G-3-P/DHAP acyltransferase under the control of a seed specific promoter.

In this example, the coding region of the G-3-P/DHAP acyltransferase found in YKR067 was inserted into a plant transformation vector RD400. The acyltransferase gene was placed under the control of the seed specific napin promoter from B. napus and the plasmid was used to transform Brassica plants according to standard protocols.

### Example 13

Genetic transformation of a plant with a gene encoding a G-3-P/DHAP acyltransferase under the control of a constitutive promoter.

In this example, the coding region of the G-3-P/DHAP acyltransferase found in YBL011 was inserted into a plant transformation vector RD400. The acyltransferase gene was placed under the control of the 35S promoter and the plasmid was used to transform Brassica plants according to standard protocols.

### Example 14

Genetic transformation of a plant with a gene encoding a G3-P/DHAP acyltransferase under the control of a seed specific promoter.

In this example, the coding region of the G-3-P/DHAP acyltransferase found in YBL011 was inserted into a plant transformation vector RD400. The acyltransferase gene was placed under the control of the seed specific napin promoter from B. napus and the plasmid was used to transform Brassica plants according to standard protocols.

Sequence Listing Free Text

In the accompanying Sequence Listing, the description of SEQ ID NOS:1-4 and 9-12 includes free text in English in the <213> and <223> fields. These descriptions are, respectively, “Artificial Sequence” and “Primer”.

1. An isolated polynucleotide comprising:
a nucleotide sequence of SEQ ID NO:5 or SEQ ID NO:7, or the complementary strand of said sequence; or
a polynucleotide sequence that hybridizes under stringent conditions to the protein coding regions of SEQ ID NO:5 or SEQ ID NO:7, or the complementary strand or a fragments thereof; or
a polynucleotide sequence which, but for the degeneracy of the genetic code, would hybridize under stringent conditions to the polynucleotide sequence of SEQ ID NO:5 or SEQ ID NO:7.

2. An isolated polynucleotide according to claim 1, having at least 80% identity in terms of encoded amino acids relative to SEQ ID NO:5 or SEQ ID NO:7.

3. An isolated polynucleotide according to claim 1, having at least 90% identity in terms of encoded amino acids relative to SEQ ID NO:5 or SEQ ID NO:7.

4. An isolated polynucleotide according to claim 1, having at least 95% identity in terms of encoded amino acids relative to SEQ ID NO:5 or SEQ ID NO:7.

5. An isolated polynucleotide according to claim 1 of SEQ ID NO:5 or SEQ ID NO:7.

6. An isolated polynucleotide according to claim 1 of SEQ ID NO:5.

7. An isolated polynucleotide according to claim 1 of SEQ ID NO:7.

8. An isolated polynucleotide forming a protein coding region of a polynucleotide sequence as defined in any one of claims 1 to 7, said region encoding a protein having Glycerol-3-Phosphate acyltransferase (G-3-P) or Dihyroxy Acetone Phosphate acyltransferase (DHAP) activity.

9. A polypeptide having an amino acid sequence according to SEQ ID NO:6 or SEQ ID NO:8, or having a sequence with at least 80% identity thereto.

10. A polypeptide according to claim 9, having at least 90% identity to SEQ ID NO:6 or SEQ ID NO:8.

11. A polypeptide according to claim 9, having at least 95% identity to SEQ ID NO:6 or SEQ ID NO:8.

12. A chimeric gene comprising an isolated polynucleotide according to any one of claims 1 to 8 operably linked to a regulatory element.

13. A plant, or a descendent of a plant, or a part of a plant or descendent thereof, having a genome modified to include a chimeric gene according to claim 12.

14. A method of modifying the lipid composition of a cell comprising:
(a) introducing into a cell capable of being transformed a genetic construct comprising a first DNA expression cassette that comprises, in addition to DNA sequences required for transformation and selection in said cells, a polynucleotide according to any one of claims 1 to 8, operably linked to a transcriptional regulatory region; and
(b) recovering a cell which contains said genetic construct.

15. A method of identifying Glycerol-3-Phosphate acyltransferase (G-3-P) or Dihyroxy Acetone Phosphate acyltransferase (DHAP) genes comprising:
(a) producing a cell comprising a conditional choline auxotrophic lipid mutant, wherein growth of said mutant is inhibited by high levels of inositol, said mutant being capable of suppression by supplementation of choline to an inositol-containing medium;
(b) producing, as a second mutant, a choline transporter mutant;
(c) combining the first and second mutants to form a double mutant; and
(d) screening said double mutant with cloned DNA, modified for expression in said cell, to identify a G-3-P/DHAP acyltransferase encoded genes capable or restoring normal growth to said mutants.
